(19) United States
(12) Patent Application Publication
(10) Pub. No.: US 2016/0186154 A1
(43) Pub. Date: Jun. 30, 2016

(54) GENETICALLY MODIFIED RUMEN MICROBES FOR PRODUCTION OF ALCOHOL AND ALLIED DOWNSTREAM PRODUCTS FROM LIGNOCELLULOSIC FEEDSTOCK

(71) Applicant: Sampanna Vijayarao MUTALIK, Karnataka, Davangere (IN)

(72) Inventor: Vijayarao Sampanna Mutalik, Davangere (IN)

(21) Appl. No.: 14/907,794

(22) PCT Filed: Jan. 23, 2014
(86) PCT No.: PCT/IB14/58483

§ 371 Date: Jan. 26, 2016

(30) Foreign Application Priority Data
Jul. 26, 2013 (IN) 3338/CHE/2013

(51) Int. Cl. C12N 9/42; C12P 7/10; C12N 15/74

(52) U.S. Cl. CPC C12N 9/2437; C12N 15/74; C12P 7/10; C12Y 302/01004

(57) ABSTRACT

The present invention relates to genetically modified rumen microbes which has been transformed by inserting into it a plasmid vector containing a gene of interest, an inducible promoter etc. The modified rumen microbes is used for efficient, cost-effective and environment-friendly production of ethanol, and allied down-stream products from plant material including but not limited to agricultural and forestry processing wastes or plant based industrial waste. The process of producing ethanol from lignocelluloses using genetically modified microbes offers various advantages over existing methods that the ethanol is produced in higher yield. As the process utilizes plant material wastes that are generated in various paper and pulp industry as feedstock, the process helps in agricultural waste management.

### TECHNICAL FIELD OF THE INVENTION

The present invention relates to genetically modified microbe. More specifically, the invention relates to a genetically modified rumen microbe for an efficient, cost-effective and environment-friendly production of ethanol, and allied down-stream products from wastes including plant material but not limited to agricultural and forestry processing wastes or plant based industrial waste.

### BACKGROUND OF THE INVENTION

Ethanol is a 2-carbon alcohol with the molecular formula CH₃CH₂OH. Ethanol is also called ethyl alcohol, pure alcohol, grain alcohol or drinking alcohol. It is a volatile, flammable, colorless liquid, which is being considered as an alternative fuel and therefore, world-wide the research is going on to produce ethanol from the cheapest available source and from an easy to use and cost-effective processes. Molasses and sugarcane have been commonly used for production of ethanol worldwide but the main issue with molasses is its acute shortage in today's world. During year 2005-2006 shortage of molasses led to increased international and domestic prices of ethanol. In the year 2006, all India Distillers Association (AIDA) had projected a deficit of 11,000 lakh litres of alcohol due to non-availability of molasses affecting primary distilleries producing both hydrous and anhydrous alcohols (fuel alcohols or ethanol) from molasses adversely.

A deep study and research of sugarcane cultivation reveals that this shortage of sugarcane and molasses is periodical and will happen once in 4-5 years cycle. During this period, in order to meet the demand of ethanol, the distillery has to rely upon alternative feed stock for the production of either hydrous or anhydrous alcohol. Additionally in India due to heavy export of molasses to other countries, the price of molasses has gone up drastically in last couple of years. Since demand of ethanol is increasing day by day worldwide and it is a big challenge to cope up with the increasing demand and it is critical to look for the alternative materials that can be used for ethanol production.

Lignocellulose a structural material that comprises much of the mass of plants is composed of cellulose, hemicellulose, and lignin and is used often as a starting material to produce ethanol and the ethanol so produced is termed as “cellulosic ethanol” or biofuel. According to U.S. Department of Energy studies conducted by Argonne National Laboratory of the University of Chicago, one of the benefits of cellulosic ethanol is that it reduces greenhouse gas emissions (GHG) by 85% over reformulated gasoline. The plant materials such as agricultural and forestry processing wastes or plant based industrial waste such as waste from paper pulp industry are the main source of lignocellulose and can be used as important material and energy source for ethanol production. However if untreated they can pose a danger to the environment and potentially valuable resources. In the recent years an increasing interest shown by the textile, food, feed & pulp and paper industries in the microbial and enzymatic processes has triggered in-depth studies of lignocellulolytic microorganisms and their enzymes. The biggest advantage of using lignocellulose is that, as a raw material it is plentiful in nature and especially cellulose is present in every plant cell wall. It is estimated that 323 million tons of cellulose containing raw materials that could be used to create ethanol are thrown away each year in US alone. So utilization of lignocellulose is not only cost-effective and efficient for production of ethanol but it also has vital role to play in agricultural waste management and it is an eco friendly process of ethanol production.

Production of ethanol from plant material has primary advantage that it is available in abundance and is diverse as a raw material compared to the other sources such as corn and cane sugars etc, but it requires a greater amount of processing to make the sugar monomers available to the microorganisms that are typically used to produce ethanol by fermentation. Lignocellulose in plant material is a combination of cellulose, hemicellulose and lignin that provides a protective sheath around the cellulose which must be modified or removed before efficient hydrolysis of cellulose can occur, and moreover the crystalline structure of cellulose makes it highly insoluble and resistant to attack. Therefore, to economically hydrolyse cellulose & hemi-cellulose more advanced pre-treatment technologies or systems in the form of genetically modified organisms are required that may be more efficient in hydrolyzing sugar monomers to produce ethanol.

The present methods and technology available to convert lignocellulose in plant material to ethyl alcohol are very costly and the higher cost of conversion is primarily due to high cost of enzymes & acids which are used for the degradation (hydrolysis) of cellulose. The cost of enzymes per litre of anhydrous alcohol produced is around 12-13 rupees, considering the high cost of ethanol production from plant material through existing routes of acid & enzyme hydrolysis there is a need to have an environment-friendly and cost-effective system that can be efficiently used for ethanol production from plant material including not only agricultural and forestry waste but also plant based industrial waste in order to fulfill the increasing demand of ethanol worldwide. Further there is need to have a process for direct hydrolysis of plant material without being pre-treated with acid or enzymes so that overall cost of the process can be minimized.

The commonly used methods at present to produce ethanol from lignocellulosic material are as follows

Separate Hydrolysis and Fermentation:

In this method, the enzymes are used for hydrolysis of lignocellulosic feedstock but the cost of this process is very high and the process needs separate enzymes for fermentation of pentose and hexose sugars. The cost of the process even rises due to high cost of pre-treating feedstock and therefore this process is not cost-effective.

Simultaneous Saccharification and Fermentation:

In this method, due to need to have enzyme cellulose, cost of production is high and it needs separate enzymes for fermentation of pentose and hexose sugars.

Simultaneous Saccharification and Co-Fermentation:

In this process, the enzymes are added to hydrolyse hemicelluloses and cellulose fractions separately. Cost of overall production of ethanol is high because of pre-treatment cost of the feedstock and separate hydrolysis requirements for hydrolysis of hemicelluloses and cellulose fractions.

Consolidated Bio-Processing:

This method involves engineering naturally occurring cellulolytic microorganisms to improve product-related properties, such as yield and titer, and engineering non-cellulolytic organisms that exhibit high product yields and titers to express a heterologous cellulase system enabling cellulose utilization. Consolidated bio-processing method is still in nascent stage and yet to be established but cost of ethanol production is high due to need to pre-treat feedstock.

Use of Microorganisms:

This method involves microorganism including different species of bacteria and fungi for production of ethanol from lignocellulose. These microorganism posses enzymes which lead to degradation of lignocellulose contained in woody plant materials. Lignocellulose gets converted to simple and complex sugars which are then converted to ethanol. The problem with this kind of method is that some of the microorganism is incapable of degrading the complex sugars, hence pre-treatment with enzymes is required which increases the overall cost of the process.

There are major drawbacks in the existing technologies used in the state of the art for production of ethanol from lignocellulose such as the cost of enzymes used for hydrolysis is very high, microorganisms is not enough for hydrolysis of both pentose and hexose sugar of lignocellulosic feedstock, before enzyme hydrolysis is done chemical pre-treatment of lignocelluloses is required and the cost of acid and alkali treatment is extremely high. Moreover because of the corrosive nature of acids all the vessels, fermenters etc to be used in the process are required to be constructed using stainless steel material which in turn increases the cost.

Considering the existing methods or technologies available in the state of the art for producing ethanol from lignocellulose there is a need of a process which may produce ethanol from lignocellulose in a high yield and is capable of hydrolyzing both pentose and hexose sugar, so that separate procedure for producing ethanol from hexose and pentose sugar is not required. Also there is a need of a process which produces ethanol from lignocellulose by direct hydrolysis without the need of pretreatment with acids or enzymes that will reduce the overall production time and at the same time make the entire process inexpensive.

In the state of the art various microorganism like Escherichia coli, Klebsiella, Oxytoca, and Zymomonas mobilis has been engineered to be used for production of ethanol but the major drawback with these organism is that they are not able to hydrolyse both pentose and hexose sugar. In the past ruminoccocus albus has been used to produce ethanol from lignocellulose successfully but with the increasing demand of ethanol as a biofuel in today's world, there is a need of a more efficient process which can produce ethanol with higher yield.

### SUMMARY OF THE INVENTION

The present invention relates to genetically modified microbe. More specifically, the invention relates to a genetically modified rumen microbes for an efficient, cost-effective and environment-friendly production of ethanol, and allied down-stream products from wastes including plant material but not limited to agricultural and forestry processing wastes or plant based industrial waste.

Another aspect of the present invention is to employ genetically modified ruminoccocus albus for producing ethanol in high yield from lignocellulose available in plant material which can be gathered from various sources like agricultural waste, forestry waste, industrial waste including plant material like waste produced by paper and pulp industry.

Yet another aspect of present invention rumen microbes has been extracted from animals like cow, sheep, chinkara and blackbuck which have capability to degrade and digest cellulosic material contained in plant. These microbes are screened for gene responsible for hydrolysis of cellulose and hemicelluloses to simple sugars and alcohol. The endocellulose and exocellulose coding genes from the donor organism is cloned with a plasmid vector pBAD202/D. The obtained plasmid vector is inserted in ruminoccocus albus and this genetically modified microbe is used for production of ethanol in higher yield.

Yet another aspect of present invention is to provide environment-friendly, cost-effective and efficient process of production of ethanol as the invention does not require pre-treatment of the plant material before hydrolysis which minimizes overall cost of operation and produces ethanol in higher yield. The present invention also has a vital role to play in managing the agricultural waste as lot of waste generated from paper and pulp industry is being utilized for production of ethanol by this process.

### DETAILED DESCRIPTION OF THE INVENTION

In order to more clearly and concisely describe and point out the subject matter of the claimed invention, the following definitions are provided for specific terms which are used in the following written description.

By the term “genetically modified microbe” we mean microbe whose genetic material is altered using genetic engineering techniques.

By the term “ruminant animals” we mean a mammal that digests plant-based food by initially softening it within the animal's first compartment of the stomach, principally through bacterial actions, then regurgitating the semi-digested mass, now known as cud, and chewing it again.

By the term “rumen microbes” we mean microbes that are extracted from the rumen of ruminant animals.

By the term “plant material” we mean agricultural waste, forestry waste, industrial waste including waste produced by paper and pulp industry.

By the term “delignification” we mean process of removal of the structural polymer lignin from plant tissue.

By the term “feedstock” we mean raw material in this case plant material which is fed into a process as input to achieve desired output.

By the term “de novo sequencing” we mean method of predicting biological feature of an organism in this case we are sequencing rumen microbes for identifying gene responsible for hydrolysis of cellulose and hemicelluloses.

By the term “plasmid vector” we mean plasmid which is a DNA molecule, which is cloned to a vector that contains the expression mechanism that is further used for genetically altering the microbe.

The present invention relates to a genetically modified anaerobic rumen microbes that are utilized for production of ethanol, and allied down-stream products, such as acetic acid, butyric acid, lactic acid, volatile fatty acids from plant materials such as agriculture and forestry waste and plant based industrial waste.

In accordance with preferred embodiments, the present invention relates to genetically modified rumen microbe that has been extracted from ruminant animals such as chinkara, black buck, buffalo, sheep etc. The microbe is transformed by inserting into it a plasmid vector containing a gene of interest, an inducible promoter etc. The present invention further relates to a process of producing ethanol, using genetically modified rumen microbes from lignocelluloses that results in higher yield of ethanol.

Another preferred embodiment of present invention, the rumen microbes that are genetically modified, have been extracted from the efficient digestive systems of the herbivorous ruminants such as chinkara, black buck, buffalo, sheep etc. Such herbivorous animals have been specifically taken for the studies as they are well-known to have cellulose and hemicelluloses degrading microbes in their digestive system. The unique feature of digestive system of ruminant animals is that they digest plant-based food by initially softening it within the animal's first stomach, then regurgitating the semi-digested mass, now known as cud, and chewing it again. The process of re-chewing the cud to further break down plant matter and stimulate digestion is called “ruminating”.

In yet another preferred embodiment of present invention, it was found that the rumen microbes are known to play a vital role in the degradation of the cellulosic plant material to simple sugars and further to all the volatile fatty acids such as acetic acid, butyric acid, lactic acid etc. Rumen microbes have various advantages as they are efficient in degradation of both hexose and pentose sugars. Secondly, one of the major issues with utilization of plant material is delignifiction of the lignocellulose, but the rumen microbes were found to be efficient enough to remove lignin easily in a very short period of time. Since there is no need to pre-treat lignocellulosic feedstock, the overall cost of the process is low.

Another preferred embodiment of present invention offers various advantages over the existing prior art that the use of genetically modified anaerobic rumen microbes enhance ethanol yield significantly. The process does not use any enzymes and feedstock need not be pre-treated with acid, hence not only the cost of overall process is low, but also it is environment friendly, as it eliminates the need of using enzymes, which sometimes can be hazardous. Any type of cellulose and hemicellulose wastes can be used as the initial raw material. Single fermenter can be utilized for fermentation of both pentose and hexose sugars, as the genetically modified rumen is capable of producing ethanol from both hexose and pentose sugar in one single process. With little modification in the process, the existing distilleries which operate on molasses/grain as the feedstock can be easily operated with plant material as a source of lignocellulose. By-product such as acetic acid was further separated using available separating techniques and through esterification route it was further converted to ethanol.

In order that this invention to be more fully understood the following preparative and testing examples are set forth. These examples are for the purpose of illustration only and are not to be construed as limiting the scope of the invention in any way.

### Example 1

### Preparing Animals for Drawing Rumen Flora

The process of production of ethanol from plant material using genetically modified rumen microbe begin with preparing the selected herbivorous ruminant animals for drawing rumen micro flora. The animals used for extraction of the microbes were fed for at least 20-25 days on the specific ratio of plant material (maize, jowar trashes, paddy grass, sugarcane trashes etc) and food concentrate. During the first week, the plant material was fed along with cattle feed concentrate to the animals and later on the plant material quantity was increased and the concentrate quantity was reduced. During last week, the animals were fed only with the desired plant material and no concentrate was added. The purpose of this specific diet was to acclimatize the micro flora of the animals with plant material so that they are more efficient in degradation of plant material. Cow, chinkara, black buck and sheep were found to be efficient ruminants. The microbes identified and utilized for cellulose, hemi-cellulose and lignin degradation are: Prevotella Sp. (Bacteria)
Fibrobacter succinogenes (Bacteria)
Ruminococcus Sp. (Bacteria)
Butyrivibrio fibrosolveus (Bacteria)
Clostridium species (Bacteria)
Eubacterium species (Bacteria)
Neo callimastix (Fungi)
Piromyces ban & Kudo (Fungi).

The rumen microbes were genetically modified in order to obtain significantly high yield of ethanol. Through various preliminary experiments it is found that all these 8 microbes can very well hydrolyse lignocellulosic feed stocks to form simple sugars and volatile fatty acids. Further through separation techniques, the sugars are separated from volatile fatty acids and are further used for fermentation & distillation to produce ethanol. The genetic modification of these microbes is done only to increase the efficiency of hydrolysis and in turn to increase the percentage of ethanol.

### Example 2

### Process of Genetic Modification of Rumen Microbes

The process followed for Genetic modification is, that all the eight organisms selected after preliminary screening in the laboratory have been subjected to De-Novo sequencing with the help of Next Generation Sequencing (NGS). NGS is massively parallel high throughput sequencing which can generate millions of reads in one sequencing run. De-Novo sequencing is possible as NGS does not require cloning and also allows very high resolution for confident base calling for all applications including for Prokaryotes and Eukaryotes that includes whole genome sequencing. 1×1 million Agilent capture array comprising 60 mer tailing probes has been designed for capturing whole genome sequencing of all the organisms. 70b flanking regions will be added upstream and downstream to each gene. The overlapping regions will be merged into a unique target region. Known repeat regions and gaps overlapping with the unique target region will be identified. Probes will not be designed in these repeat regions, however probes will be allowed to have a maximum of 10 bases into repeat regions to avoid losing coverage on borders. Illumine/Solexa specifically (GAIIx) will be used for De-Novo sequencing. Once the whole genome sequencing of all the organisms is completed, the functional genes will be located and function of each gene will be predicted. More specifically gene responsible for the hydrolysis of cellulose and hemicelluloses to simple sugars and alcohol will be predicted. The genes will be cloned to suitable aerobic host with copy number rise to increase the efficiency of the production of alcohol.

### Example 3

### Preparation of Plasmid Vector

Cloning of A2 endocellulose and exocellulose genes with the size of 1500b and 1546 b is done using BAD Directional TOPO® Expression kit. The pBAD Directional TOPO® Expression Kit utilizes a highly efficient, 5-minute cloning strategy (“TOPO® Cloning”) to directionally clone a blunt-end PCR product into a vector for soluble, regulated expression and simplified protein purification. Blunt-end PCR products clone directionally at greater than 90% efficiency with no ligase, post-PCR procedures, or restriction enzymes required. In addition, pBAD202/D-TOPO® vector contains the His-Patch (HP) thioredoxin leader for increased translation efficiency and solubility of recombinant fusion proteins. Expression in E. coli is driven by the araBAD promoter (PBAD). The AraC gene product encoded on the pBAD202/D-TOPO® vector positively regulates this promoter.

### Example 4

### Preparation of Competent Cell

A competent cell was prepared by adding 3 μL of the TOPO® Cloning reaction from Performing the TOPO® Cloning Reaction into a 0.1 cm cuvette containing 50 μL of electrocompetent cells and was mixed gently. Mixing by pipetting up and down is avoided, formation of bubbles should not take place. Samples are electroporated using electroporator. Immediately 250 μL of room temperature S.O.C. medium is added to the cuvette. Then the solution is transferred to a 15 mL snap-cap tube (e.g. Falcon) and shaked well for at least 1 hour at 37° C. to allow expression of the antibiotic resistance gene. 50-100 μL from each transformation was spread on a pre warmed selective plate and incubated overnight at 37° C. It is recommended that two different volumes should be plated to ensure that at least one plate will have well-spaced colonies. An efficient TOPO® Cloning reaction produced several hundred colonies. 5 colonies were picked for analysis. After analysis suitable clones of the cess were used for broth culture.

### Example 5

### Process of Obtaining Ethanol Using Genetically Modified Microbes

The process of obtaining alcohol from lignocelluloses was carried out under anaerobic conditions and it comprised of following steps:
Plant material was chopped and visible impurities of plastic or metal were removed.
Chopped plant material was added to a conical flask. Since genetically modified rumen microbes were used in the process of lignocellulose degradation, pre-treatment of the plant material was not required and sterile water was added directly to the plant material.
Mixture of genetically modified rumen microbes was added to the mixture of plant material followed by incubation for specified time to allow hydrolysis of the lignocellulose to hexose and pentose sugars.
After hydrolysis, the contents of the flask are filtered into another conical flask and the pH was adjusted to 4-4.5. Industrial yeast (12 gram) was added to the flask, the flask was cotton plugged and kept at room atmosphere.
The fermentation was carried out for 32-36 hours and thereafter liquid was taken for lab-distillation.
The repeated hydrolysis and fermentation of the same plant material was carried out to make sure complete degradation and utilization of the same.

Alcohol percentage obtained using genetically modified microbes was found to be at least 4.5-6.0% v/v compared to controls that yield 2.2-2.4 v/v. The advantages of genetically modified rumen is reduction in the hydrolysis period from 36-40 Hrs to 18-24 hrs and consistency in the results when compared to natural Ruminococcus. The significant efficiency of the genetically modified rumen microbes can be seen in the table given below.

TABLE 1
show the comparative results obtained by using naturally occurring
rumen microbes and genetically modified rumen microbe.

| | | | | Genetically |
|---|---|---|---|---|
| | | Before Microbial | Natural | modified |
| Sl. No. | Parameters | Degradation | Rumen | organism |
| 1 | Alpha Cellulose (%) | 39.24 | 24.19 | 20.48 |
| 2 | Holocellulose (%) | 71.01 | 52.65 | 46.45 |
| 3 | Pentosans (%) | 20.25 | 18.19 | 17.68 |
| 4 | Other sugars (%) | 11.52 | 10.27 | 10.10 |
| 5 | Lignin (%) | 16.52 | 14.66 | 13.28 |

| | | | |
|---|---|---|---|
| 6 | Ethanol by hydrometer (%) | 2.24 | 2.56 |

The optimization of various parameters was done in order to standardize the condition for maximum production of ethanol. The hydrolysis was carried out at various pH range and the ethanol production was tested at various time intervals from the time of starting the process of hydrolysis using various feedstocks like Cornstover and Trash, Sorghum stover and trash, Paddy straw etc., as a starting material. After 4 hours of addition of microbes to the feedstock (in an anaerobic condition) the formation of sugars and volatile fatty acids starts and are continuously separated from the hydrolysis reactor for further separation and for future process steps like fermentation and distillation. On repetitive hydrolysis the left out cellulose & hemicellulose fractions in the feedstock at the end will be very low.

It is to be understood, however, that even though numerous characteristics and advantages of the present invention have been set forth in the foregoing description, together with details of the structure and function of the invention, the disclosure is illustrative only. Changes may be made in the details, especially in matters of shape, size, and arrangement of parts within the principles of the invention to the full extent indicated by the broad general meaning of the terms in which the appended claims are expressed.

The genetically modified organism i.e. genetically modified Ruminococcus albus relating to this invention was submitted in MTCC Chandigarh on 14 Jun. 2013 recorded as deposition date and has been assigned an accession number MTCC 5834. Kindly accept the same as a part of specification that better describes the invention.

I claim:

1. A genetically modified ruminoccocus albus comprising:
a) a gene responsible for hydrolysis of cellulose and hemicelluloses;
b) a inducible promoter; and
c) a mobilisable plasmid which is cloned to a vector.

2. The genetically modified microbes according to claim 1 wherein said microbe is isolated from a group of animals like cow, chinkara, blackbuck, sheep etc.

3. The genetically modified microbe according to claim 1 wherein said promoter is araBAD.

4. The genetically modified microbe according to claim 1 wherein said plasmid is of size 4448 without insert and of size 7494 with insert.

5. The genetically modified microbe according to claim 1 wherein said plasmid vector contains the his-patch (HP) thioredoxin leader for increased translation efficiency and solubility of recombinant fusion proteins.

6. The genetically modified microbe according to claim 1 wherein said plasmid vector contains AraC gene product which positively regulates said promoter.

7. A genetically modified microbe according to claim 1 wherein the said microbe is prepared by a process comprising steps as below:
a) isolating rumen microbes from a group of ruminant animals;
b) identifying cellulose and hemicelluloses degrading genes in said microbes;
c) preparing plasmid vector to be cloned to said genes;
d) inserting said plasmid vector into the ruminoccocus albus.

8. The genetically modified microbe produced by a process according to claim 7 wherein said genes are identified by de novo sequencing.

9. A method of producing ethanol from lignocelluloses using a genetically modified microbe, comprising:
a) mixing feedstock and genetically modified microbe in a apparatus;
b) incubating the mixture for specified time to allow hydrolysis of the lignocellulose to hexose and pentose sugars;
c) fermenting the content obtained after hydrolysis for about 32-36 hours in presence of industrial yeast and at PH of 4-4.5;
d) repeating hydrolysis and fermentation step several times for complete degradation of lignocelluloses.

10. The method of claim 9 wherein said feedstock is selected from a group of cornstover, trash, sorghum, stover, paddy straw etc., as a starting material.
